(19) United States
(12) Patent Application Publication
(10) Pub. No.: US 2004/0171957 A1
(43) Pub. Date: Sep. 2, 2004

(54) Method of attenuating the influence on UWB communications of interference produced by bursty radio transmission systems

(76) Inventors: John Farserotu, Neuchatel (CH); Andreas Hutter, Neuchatel (CH); Johannes Gerrits, Cortaillod (CH); Alexandre Pollini, Yverdon (CH)

Correspondence Address: JACOBSON HOLMAN PLLC, 400 SEVENTH STREET N.W., SUITE 600, WASHINGTON, DC, 20004, US

(21) Appl. No.: 10/787,767

(22) Filed: Feb. 27, 2004

(30) Foreign Application Priority Data
Feb. 28, 2003 (FR) 03 02475

(51) Int. Cl. A61B 5/04

(52) U.S. Cl. 600/509

(57) ABSTRACT

Useful information is transmitted by means of a UWB signal from a sender comprising a UWB signal modulator and a send antenna to a receiver comprising a receive antenna and detection and demodulation means for discriminating the useful information in the signal received at the receive antenna. In the receiver, the signal received at the receive antenna is compared to a first power threshold value and transmission of the received signal to the detection/demodulation means is blocked if the power level of the received signal exceeds the first threshold value.

### BACKGROUND OF THE INVENTION

1. Field of the Invention

The present invention relates to a method of reducing the influence on an ultra wide band (UWB) communication system of interference caused by existing wireless communication systems.

The invention aims more particularly to reduce or even avoid collision with a neighboring bursty wireless transmission system that might impede good reception of a UWB signal for communicating, especially over short distances, binary information whose bit rate is relatively low, for example of the order of 10 kbit/s (as in personal area network (PAN) type communications).

2. Description of the Prior Art

To convey data to be transmitted, the prior art “pulsed” radio communication technique can use a carrier in the form of pulses of short duration and wide frequency band, also known as “wavelets”, containing the information in the form of modulation of the relative positions of the wavelets in time (i.e. PPM modulation), for example. The bandwidth of the signal generated can be up to 30% of the center frequency, which can be in a range from 1 to 5 GHz, for example.

One potentially interesting application of the UWB technique is to communication in environments (for example commercial or utilitarian buildings, such as hospitals), in which different wireless communication links (GSM, UMTS, WLAN, WLL, Bluetooth™, etc. links) exist already on different frequency bands and with a high power density. As a general rule, these links use spread spectrum techniques and have a range and bit rates much higher than UWB signal transmission techniques. For example, WLAN type communications have a range up to 30 m and bit rates from 2 to 50 Mbit/s.

Although, for a given communication link, UWB communication techniques have the advantage of enabling distribution of the transmitted power over a very wide band of frequencies, so that they are less of a nuisance to other, narrowband, communication links, they are nevertheless subject to interference from existing radio links that is perceived at the receiver as interference degrading good transmission of wanted messages.

An object of the invention is to provide a method of minimizing the influence on UWB transmissions of interference caused by existing radio communications.

### SUMMARY OF THE INVENTION

The invention therefore provides a method of attenuating the influence of interference produced by bursty radio transmission systems on UWB communications during which useful information is transmitted by a signal from a sender comprising a UWB signal modulator and a send antenna to a receiver comprising a receive antenna and detection and demodulation means for discriminating the useful information in the signal received at the receive antenna, which method consists in, in the receiver, comparing the signal received at the receive antenna to a first power threshold value and blocking transmission of the received signal to the detection/demodulation means if the power level of the received signal exceeds the first threshold value.

The method of the invention can also have the following advantageous additional features:

it consists in comparing the received signal to a second power threshold value and authorizing processing of the received signal by the detection/demodulation means if the power level of the received signal is below the second threshold value;

if the level of the received signal is between the first and second threshold values, periods of silence present in components of the signal caused by interference are detected in the received signal, a temporal adjustment signal is generated, and, in the sender, the temporal characteristics of the useful information are adjusted to provide communication between the sender and the receiver only during the periods of silence;

the second threshold value is adjustable from outside the receiver;

the second threshold value is set by the receiver itself on the basis of signals received;

the temporal adjustment signal is transferred from the receiver to the sender by means of a UWB signal;

the temporal adjustment signal is adjusted as a function of the evolution in time of the periods of silence;

a warning signal is generated in the receiver when transmission of the signal to the detection/demodulation means is blocked;

a warning signal is generated in the receiver when the level of the received signal is between the first and second threshold values.

Thanks to these features, communication between the sender and the receiver is inoperative when the interference is at a level such that the useful information cannot be received reliably.

The invention also provides a system for implementing the method of the invention, comprising in combination a sender adapted to send the UWB signal, a receiver adapted to receive the UWB signal, equipment for detecting a physiological parameter adapted to generate the useful information and connected to the sender, the equipment being adapted to be worn by a person and to detect a physical characteristic thereof, and a relay station connected to the receiver and adapted to transfer a signal representative of the received signal for medical monitoring purposes.

The system of the invention can also have the following advantageous additional features:

the relay station is adapted to be worn by the medically monitored person;

the relay station is a fixed station connected to a long-range network.

Other features and advantages of the present invention will become apparent in the course of the following description, which is given by way of example only and with reference to the accompanying drawings.

### BRIEF DESCRIPTION OF THE DRAWINGS

FIG. 1 is a block schematic of a UWB sender and a UWB receiver that communicate with each other using wavelet signals and the method of the invention.

FIG. 2 is a timing diagram depicting one important aspect of the method of the invention.

### DETAILED DESCRIPTION OF THE PREFERRED EMBODIMENT

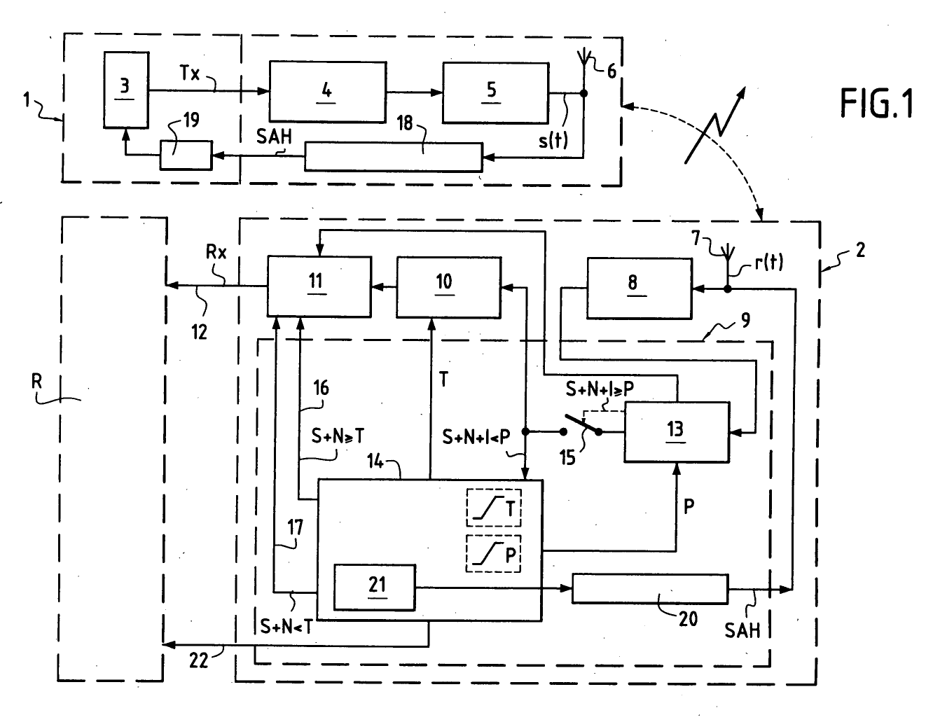

FIG. 1 shows a sender 1 and a receiver 2 between which communication can be established over a certain distance by means of UWB signals.

Although the invention is not limited to it, one interesting application of the invention is to personal area networks (PAN), for example to monitoring the state of health of persons suffering from illnesses where it is very important to know certain physiological data at all times. This might apply, for example, to persons suffering from heart disease, in which case an essential item of data is the heart rate.

The physiological data having been detected, the corresponding information can be transmitted to a nearby relay station (at a distance of a few meters, for example) at a low bit rate, for example of the order of only 5 to 10 kbit/s.

Thus in this application, which is described by way of example only, the sender 1 can be associated with or be part of an equipment 3 for detecting a physical characteristic of a person, which characteristic is important for medical monitoring of the person. In the specific example described here, the equipment is for detecting the heart rate, and is located for this purpose on the body of a patient to be monitored, at a place where the circulation of the blood can be easily examined. For example, the whole of the sender 1 and the equipment 3 can be installed on the ear of the patient to detect the circulation of the blood in situ. The receiver 2 can be situated in a wristwatch worn by the person and serving as the relay station. The information collected can be forwarded via the relay station, for example to a medical monitoring center, possibly via a telephone or a long-range network such as the Internet.

In a different embodiment, the receiver 2 could be incorporated directly into a telephone which then serves as the relay station. The relay station is symbolically represented by the rectangle R in FIG. 1.

Referring to FIG. 1, the equipment 3 generates a stream of pulses Tx in which the useful information (in this instance the heart rate) can be contained in the form of PPM modulation, familiar to the person skilled in the art. The stream of pulses is applied to the sender 1, which includes a UWB modulator 4 connected for this purpose to the equipment 3, the modulated signal being applied to a filtering and formatting circuit 5 sending to an antenna 6 a UWB useful signal s(t) that is PPM modulated as a function of the signal Tx.

The signal s(t) transmitted by the antenna 6 is received by an antenna 7 of the receiver 2, and is subject to interference, such as noise, fading or interference caused by other communications in progress nearby. The signal is therefore of the form r(t)=S/(N+I), in which S is the power received at the antenna 7, N represents noise and I represents interference.

The antenna 7 is connected to a filter 8 which transmits the received and filtered signal to a processing unit 9 adapted to reduce or even eliminate the influence of the interference on the signal r(t).

After processing, the signal is fed to a UWB detector 10 and from there to a UWB demodulator 11 which produces at its output data Rx whose content is representative of the signal Tx previously sent to the UWB modulator 4 of the sender 1. The data Rx appears at an output terminal 12.

The processing unit 9 comprises protection means 13 providing protection against interference and detection means 14 to detect interference and then to analyze its content.

The detection unit 14 is adapted to set two power threshold values P and T where P>>T.

The threshold value P is applied to the protection unit 13. The latter is adapted to compare the power of the filtered signal appearing at the output of the filter 8 to the threshold P. If the level of the signal is greater than or equal to that threshold, the protection unit 13 commands a blocking function symbolized by the switch 15 in FIG. 1, inhibiting any transmission of the signal to the detector 10. In this case, the power of the interference is such that correct reception of the useful signal is not possible, so that it is necessary to prevent the receiver 2 from operating. A warning signal can be generated by the protection unit 13 and transmitted to the demodulator 11, so that the reception of the high level of interference can be signaled at the output 12 and from there to the relay station R. In the contrary situation, the blocking function is cancelled (the switch 15 is closed) and the filtered signal is applied to the detector 10 and also to the detection unit 14.

Thanks to another important feature of the invention, it is possible to discern the useful signal Rx in the received signal r(t) even in the presence of one or more sources of interference, provided that their level is not above the threshold value P.

If this is the case, the switch 15 is closed. The power level of the filtered signal r(t) is then compared to the second threshold value T. If the power of the filtered signal is greater than or equal to that of the threshold T, the useful information cannot be detected correctly in the filtered signal. In this case, the detection unit 14 sends a rejection signal via a line 16 to the demodulator 11, which is therefore disabled. Under these conditions also, no useful signal is supplied to the output 12.

However, the sender 1 also includes a receiver circuit 18 that is connected to a clock circuit 19 determining the temporal relationship of the useful pulses of the signal Tx supplied by the equipment 3.

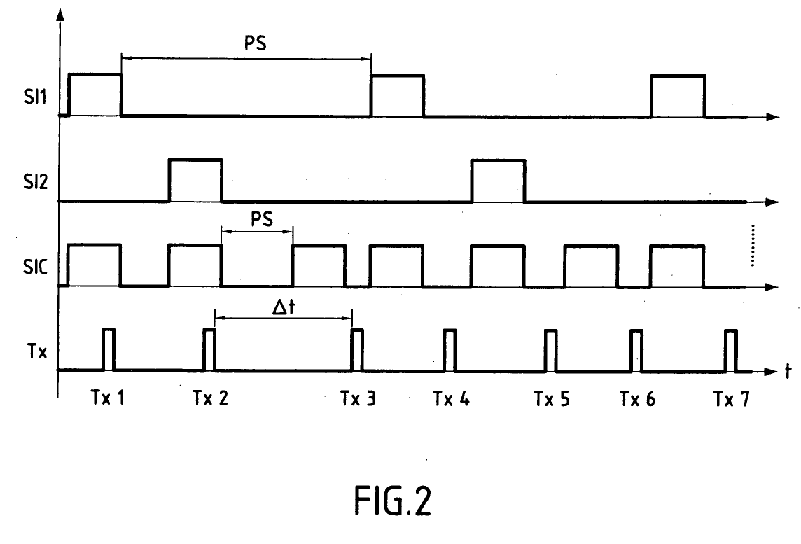

The receiver 2 further includes a sender circuit 20 that is adapted to send to the antenna 7 a clock adjustment signal SAH that contains clock adjustment information and can be transmitted to the sender 1, in which it is received by the receiver circuit 18. The detection unit 14 is adapted to analyze interference component(s) in the signal received by the antenna 7 and discriminate in each of them periods of silence PS between periods of activity (see also FIG. 2). The detection block 14 can derive from this discrimination information the periods of silence PS during which there are no components caused by the interference sources in the signal r(t) received at the antenna 7. The detection unit 14 also includes a generator 21 of the adjustment signal SAH that is modulated onto a carrier by the sender circuit 20 and sent to the receiver circuit 18 via the antennas 7 and 6. The adjustment signal SAH is representative of a time Δt by which the useful signal Tx from the equipment 3 will be delayed so that the information in the received signal r(t) to be detected and sent to the output 12 no longer coincides with all the pulses caused by interference.

The above process is depicted in FIG. 2, in which the first two lines represent two signals SI1 and SI2 from sources of interference, the third line of the FIG. 2 diagram representing the image of a composite disturbing signal SIC resulting from the various interference signals. In the example shown, it is assumed that the two useful pulses Tx1 and Tx2 coincide with pulses caused by interference, with the result that they are neither demodulated nor sent to the output 12. Thus in this situation the detection unit 14 sends an inhibition signal on the line 16.

As soon as the detection unit 14 has determined the periods of silence PS in all the interfering transmissions, it sends the adjustment signal SAH to the sender 1 and simultaneously enables the demodulator 11 by activating the line 17. The subsequent useful pulses Tx3, Tx4, Tx5, etc. can then be accepted and are transmitted to the output 12. If the length of the period of silence PS changes, the detection unit 14 varies the interval Δt accordingly so that the useful pulses Tx are always sent during a period of silence PS if the power of the received interference is between the threshold values P and T.

On the other hand, if the power of the filtered signal is below the second threshold value T, the detection unit 14 sends an enabling signal to the demodulator 11 via the line 17, with the result that the signal detected by the detector 10 is processed directly by the demodulator 11 and a useful signal Rx can be placed at the output 12, provided, of course, that a signal s(t) is sent from the sender 1 at the time concerned.

The value of the power threshold T must be set as a function of the characteristics of the interference sources liable to transmit in the space in which the sender 1 and the receiver 2 are used and also as a function of the level of noise that may arise in that space. The value is therefore preferably either adjustable from outside the receiver or set by the receiver itself. The receiver can set this value by allowing the detector 14 to “listen” for a certain time to its radio environment. This automatic detection of the reference threshold values can be repeated at will at appropriate time intervals. In any event, the threshold value must be relatively low to avoid detection errors caused by sources of interference and sufficiently high to prevent false alarms.

All operating states such that the crossing of the values P and T and the temporal offsetting by means of the signal SAH can advantageously be signaled to the relay station R by means of a signaling function implemented in the detection unit 14, the corresponding signals being applied to the relay station R via a line 22.

There is claimed:

1. A method of attenuating the influence of interference produced by bursty radio transmission systems on UWB communications during which useful information is transmitted by a signal from a sender comprising a UWB signal modulator and a send antenna to a receiver comprising a receive antenna and detection and demodulation means for discriminating said useful information in the signal received at said receive antenna, which method consists in, in said receiver, comparing said signal received at said receive antenna to a first power threshold value and blocking transmission of said received signal to said detection/demodulation means if the power level of said received signal exceeds said first threshold value.

2. The method claimed in claim 1, further consisting in comparing said received signal to a second power threshold value and authorizing processing of said received signal by said detection/demodulation means if said power level of said received signal is below said second threshold value.

3. The method claimed in claim 2 wherein, if the level of said received signal is between said first and second threshold values, periods of silence present in components of said signal caused by interference are detected in said received signal, a temporal adjustment signal is generated, and, in said sender, the temporal characteristics of said useful information are adjusted to provide communication between said sender and said receiver only during said periods of silence.

4. The method claimed in claim 2 wherein said second threshold value is adjustable from outside said receiver.

5. The method claimed in claim 2 wherein said second threshold value is set by said receiver itself on the basis of signals received.

6. The method claimed in claim 3 wherein said temporal adjustment signal is transferred from said receiver to said sender by means of a UWB signal.

7. The method claimed in claim 3 wherein said temporal adjustment signal is adjusted as a function of the evolution in time of said periods of silence.

8. The method claimed in claim 1 wherein a warning signal is generated in said receiver when transmission of said signal to said detection/demodulation means is blocked.

9. The method claimed in claim 3 wherein a warning signal is generated in said receiver when the level of said received signal is between said first and second threshold values.

10. A system for implementing a method as claimed in claim 1, comprising
in combination a sender adapted to send said UWB signal and a receiver adapted to receive said UWB signal,
an equipment for detecting a physiological parameter adapted to generate said useful information and connected to said sender, said equipment being adapted to be worn by a person and to detect a physical characteristic thereof, and
a relay station connected to said receiver and adapted to transfer a signal representative of said received signal for medical monitoring purposes.

11. The system claimed in claim 10 wherein said relay station is adapted to be worn by the medically monitored person.

12. The system claimed in claim 10 wherein said relay station is a fixed station connected to a long-range network.

13. The system claimed in claim 10 wherein said physical characteristic is the heart rate.
